(19) United States
(12) Patent Application Publication
(10) Pub. No.: US 2021/0299226 A1
(43) Pub. Date: Sep. 30, 2021

(54) METHOD OF TREATING AND PREVENTING CORONAVIRUSES WITH LOW DOSAGE INTERFERON BETA-1A AS A TH17 INHIBITOR

(71) Applicant: Claresa Levetan, Bryn Mawr, PA (US)

(72) Inventor: Claresa Levetan, Bryn Mawr, PA (US)

(21) Appl. No.: 16/835,905

(22) Filed: Mar. 31, 2020

(51) Int. Cl. A61K 38/21; A61K 9/00; A61P 31/14; A61K 31/7056; A61K 31/427; A61K 31/685; A61K 31/375

(52) U.S. Cl. CPC A61K 38/215; A61K 9/0053; A61K 9/0073; A61K 9/0043; A61K 31/375; A61K 31/7056; A61K 31/427; A61K 31/685; A61P 31/14

(57) ABSTRACT

This invention relates to methods for usages, dosing and delivery of interferon beta-1a for use in children, adults and other mammals for the prevention and treatment of coronaviruses including COVID-19 with the direct delivery of interferon beta-1a to the Th17 receptors in the mucosal surfaces of the intestinal and pulmonary tract for the prevention of Th17 downstream cytokines. This inventor uses interferon beta-1a as a Th17-inhibitor to the mucosal surfaces of the lung and intestine in low dosages of 10,000 IU and below. The invention also provides for interferon beta-la used in combination with antiviral pharmaceuticals to also benefit the prevention and treatment of coronaviruses including COVID-19.

### FIELD OF THE INVENTION

Pharmaceutical compounds and compositions are disclosed that are useful to treat and prevent the negative effects that result from coronavirus infections, including COVID-19.

### BACKGROUND OF THE INVENTION

The potential and reality of global pandemics from viral infections is an urgent matter that must be addressed. Potential vaccines and pharmaceuticals to combat the outbreak of such pandemics are needed. One such pharmaceutical product that is discussed in this application is interferon beta-1a used alone or in combination with other antiviral pharmaceutical agents or drugs, or other therapies.

Interferon beta-1a is currently used in the treatment of the autoimmune disease multiple sclerosis. In 1996, the FDA approved interferon beta-1a intramuscularly for usage among patients with multiple sclerosis at a dosage of 6,000,000 IU. Currently, interferon beta-1a is used to treat multiple sclerosis and relapsing remitting multiple sclerosis via systemic intramuscular injections at dosages of 6,000,000 IU to 12,000,000 IU.

Interferon beta-1a is a direct inhibitor of the destructive Th17 pathway. See Ramgolam V S, Sha Y, Jin J et al., IFN-b Inhibits Human Th17 Cell Differentiation, J Immunol 2009; 183:5418-5427. The mechanism of action of interferon beta-1a in multiple sclerosis is the reduction in neuron inflammation and its downstream attacking cytokines. See, Kieseier B C. The mechanism of action of interferon-(3 in relapsing multiple sclerosis. CNS Drugs. 2011; 25(6):491-502.

Likewise, it is known that COVID-19 infections trigger a Th17-mediated cytokine storm that attacks cells, tissues and organs resulting in the symptomatology, morbidity and mortality. See, Wu D, Yang X O, TH17 responses in cytokine storm of COVID-19: An emerging of JAK2 inhibitor Fedratinib, 2020 Journal of Microbiology, Immunology and Infection.).

As such, today the conventional thinking is to use high dosages of interferon beta-1a (6,000,000 IU to 12,000,000 IU), typically administered by intramuscular injection to systemically treat symptoms. It has also been found that interferon beta-1a, when tested in vitro, prevents replication of the coronaviruses SARS (Hensley L E, Fritz E A, Jahrling P B et al., Interferon Beta 1a and SARS Coronavirus Replication. Emerg Infect Dis. 2004; 10(2): 317-319.).

Despite the typical systemic delivery of interferon beta-1a being intramuscular injection, it is now known that seventy percent (70%) of the immune system resides in gastrointestinal tract. See, Vighi G, Marcucci F, Sensi L et al., Allergy and the Gastrointestinal System. Clin Exp Immunol. 2008; 153(Suppl 1): 3-6. It is also known that Th17 and its direct downstream cytokine, interleukin-17 (IL-17) are constitutively found in the mucosa of the intestinal tract and the mucosa of the respiratory tract. See, Esplugues E, Huber S, Gagliani N. Control Of Th17 Cells Occurs In The Small Intestine. Nature. 2011; 475(7357):514-8. See also, Mucida D, And Salek-Ardakani S, Regulation of Th17 Cells In The Mucosal Surfaces. J Allergy Clin Immunol. 2009; 123(5): 997-1003. See also, Jaffar Z, Ferrini M E, Herritt L A, Cutting Edge: Lung mucosal Th17-mediated responses induce polymeric immunoglobulin receptor expression by the airway epithelium and elevate secretory IgA levelslJ Immunol. 2009; 182(8): 4507-4511. See also, Caucheteux S M, Jane Hu-Li, J, Rebar Mohammed R Cytokine Regulation of Lung Th17 Response to Airway Immunization using LPS Adjuvant Mucosal Immunol. 2017; 10(2): 361-372.

In addition, given the inventor's recent investigation, it is now understood that administering high doses of interferon beta-1a orally or by inhalation or by nasal spray or drops above 10,000 IU have the undesired effect of Th17 receptor desensitization and downregulation which renders the interferon beta-1a ineffective.

As such, it would be desirable to determine methods, dosing regimens, and new modalities of delivery of interferon beta-1a to treat and prevent coronaviruses such as COVID-19, while at the same time, preventing the undesired effect of Th17 receptor desensitization.

### BRIEF SUMMARY OF THE INVENTION

Embodiments of the present invention provide novel methods, dosing regimens, and new modalities of delivery of interferon beta-1a for the treatment and prevention of coronaviruses including COVID-19.

In the first embodiment, the present invention provides a method of treating or preventing the effects of a coronaviruses infection including COVID-19, where the method utilizes administering a therapeutically effective amount orally or by inhalation or by nasal spray or drops a composition comprising a dosage of less than 10,000 IU of interferon beta-1a.

In a second embodiment, the present invention provides a method of treating or preventing the effects of a coronaviruses infection including COVID-19, where the method utilizes administering a therapeutically effective amount orally or by inhalation or by nasal spray or drops a composition comprising a Th17 inhibitor.

In a third embodiment, the present invention provides a method of treating or preventing the effects of a coronaviruses infection including COVID-19, where the method utilizes administering a therapeutically effective amount orally or by inhalation or by nasal spray or drops a composition comprising a Th17-inhibitor in combination with administering a therapeutically effective amount of an antiviral.

In a fourth embodiment, the present invention provides a method of treating or preventing the effects of a coronaviruses infection including COVID-19, where the method utilizes administering a therapeutically effective amount orally or by inhalation or by nasal spray or drops a composition comprising a Th17-inhibitor in combination with administering a therapeutically effective amount of other agents (for example, chloroquine, hydroxychloroquine, Zithromax (Z-Pak), Vitamin C, anti-inflammatories, hydrocortisone, sarilumad, etc.).

### BRIEF DESCRIPTION OF THE DRAWINGS

For a fuller understanding of the nature and advantages of the present invention, reference should be made to the following detailed description taken in connection with the accompanying drawings, in which:

FIG. 1 depicts the structure of interferon beta-1a.

FIG. 2 is a graph depicting the dosage dependent efficacy of interferon beta-1a.

### DEFINITIONS

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, “treating” or “to treat” a condition or patient refers to taking steps to obtain beneficial or desired results, including clinical results. For purposes of this invention, beneficial or desired clinical results include, but are not limited to, alleviation or amelioration of one or more symptoms of as a result of a coronavirus infection, including COVID-19, diminishment of extent of infection, delay or slow the infection progression, amelioration, palliation or stabilization of the infection, and other beneficial results described below.

As used herein, “preventing” or “to prevent” a condition or patient refers to taking steps to obtain beneficial or desired results, including clinical results. For purposes of this invention, beneficial or desired clinical results include, but are not limited to, preventing one or more symptoms of as a result of a coronavirus infection, including COVID-19.

Symptoms of a coronavirus infection, including COVID-19 include fever, tiredness, dry cough, and difficulty breathing. A coronavirus infection, including COVID-19 may be diagnosed by methods well known to one of ordinary skill in the art.

As used herein, “reduction” of a symptom or symptoms (and grammatical equivalents of this phrase) means decreasing of the severity or frequency of the symptom(s), or elimination of the symptom(s).

As used herein, an “antiviral” is a pharmaceutical agent or drug that can be used to prevent the infection the virus of or to inhibit the proliferation of a virus in any manner known in the art. Specific antiviral pharmaceutical agents or drugs referred to herein include Ribavarin, Ritonavir, and Remdesivir, however, the term “antiviral” is intended to include all pharmaceutical agents or drugs with antiviral properties presently known or unknown.

As used herein, “administering” or “administration of” a pharmaceutical agent or drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, a “subject” or “patient” is a mammal, typically a human, but optionally a mammalian animal of veterinary importance, including but not limited to horses, cattle, sheep, dogs, and cats.

As used herein, a “therapeutically effective amount” of a drug or agent is an amount of a drug or agent that, when administered to a subject with a disease or condition, will have the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

As used herein, a “therapeutically effective amount” of a drug may also be an amount of a drug that when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and may occur only after administration of a series of doses. Thus, a therapeutically effective amount may be administered in one or more administrations.

### DETAILED DESCRIPTION OF THE INVENTION

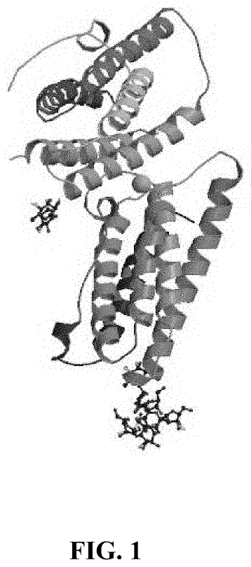

Interferon beta-1a, the structure of which is shown in FIG. 1, has been used for the treatment of multiple sclerosis in systemic intramuscular dosages in the 6,000,000 IU to 12,000,000 IU range per injection. In the last decade, in the field of multiple sclerosis, interferon beta-1a has been considered to play a role in reducing neural inflammation. See, Kieseier B C. The mechanism of action of interferon-0 in relapsing multiple sclerosis. CNS Drugs. 2011; 25(6):491-502.

In contrast to the benefits recognized by systemic administration of high doses of interferon beta-1a, a randomized study found that when a maximum of 6,000,000 IU (60,000 IU was the minimum dosage) of interferon beta-1a was safely administered orally for six months, there was safety without systemic immune suppression but no efficacy among patients with multiple sclerosis. That is, the same high dosages of interferon beta-1a that has shown efficacy when administered systemically via intramuscular injection, but not effective when presented in an oral administration. In addition, two other studies were conducted using inhaled interferon beta-1a via nebulizer in a high dosage of 6,000,000 IU daily and was also shown to be safe, but again, no efficacy in asthma was shown, See, Jackson D J., Inhaled Interferon: A Novel Treatment for Virus-induced Asthma? Am J Respir Crit Care Med.2014; 190(2):123-134. See also, McCrae C, Olsson M, Aurell M On-Demand Inhaled Interferon-Beta 1a for the Prevention of Severe Asthma Exacerbations: Results of the INEXAS Phase 2a Study Am J Respir Crit Care Med 2018; 197:A6165.

So although the systemically administering high doses of interferon beta-1a were effective in reducing symptoms the same high doses were not effective in reducing the symptoms when administered orally or by inhalation (i.e. via nebulizer or inhaler) or by nasal spray or drops. The results were disappointing because the ability to administer a therapeutically effective amount orally (via a pill, capsule, tablet, chewable tablet, gum, chew, liquid, lozenge, dissolvable, etc.) or by inhalation (via a nebulizer, inhaler, etc.) or by nasal spray or drops would be much easier for subjects or patients to accept versus painful intramuscular injections. Further, it has been found that previous inhaled, intramuscular and subcutaneous injections of interferon beta-1a of high dosages, typically in a dosage of 6,000,000 IU, can result in systemic immune suppression with many side effects including the potential for cancer and viral diseases such as Progressive Multifocal Leukoencephalopathy, which is fatal and for which there is no treatment.

Like other immune triggering events such as multiple sclerosis and asthma, it is known that COVID-19 triggers a Th17-mediated immune attack that results in morbidity and mortality.

The present invention takes a different approach from the high dosage systemic administration of interferon beta-1a as a Th17 inhibitor and instead provides a method of administering a low dosage of interferon beta-1a (less than 10,000 IU orally or by inhalation or by nasal spray or drops) to inhibit the Th17 mediated and destructive immune cascade resulting from a coronavirus infections, such as a COVID-19.

The approach of administering a low dose (less than 10,000 IU) of interferon beta-1a orally or though inhalation or by nasal spray or drops was derived from this inventor's recent understanding that dosages higher than 10,000 IU of interferons (including interferon beta-1a) results in receptor desensitization and down regulation of such receptors because interferons are delivered directly to the Th17 and IL-17 receptors in the mucosal surface of the intestinal and respiratory tracts. It is important to remember that seventy percent (70%) of the immune system resides in gastrointestinal tract and Th17 and its direct downstream cytokine, interleukin-17 (IL-17) are constitutively found in the mucosa of the intestinal tract and the mucosa of the respiratory tract. Thus, new to the art, this investigator finds that delivery of oral or inhaled interferons in dosages above 10,000 IU have no efficacy in limiting the detrimental effects of the Th17 cascade because higher dosages lead to Th17 receptor desensitization and downregulation.

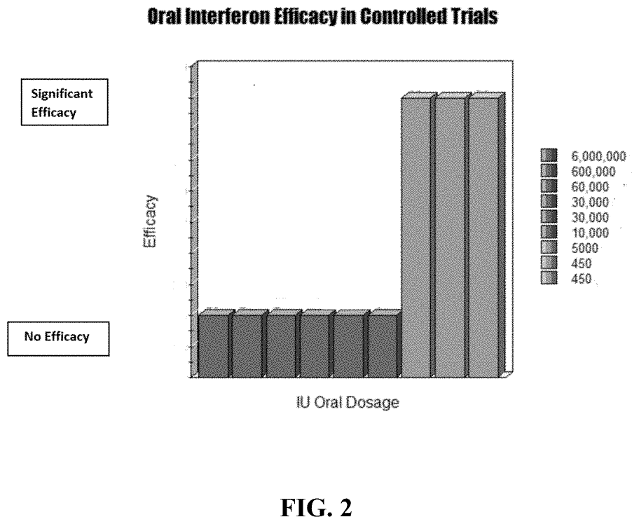

To that end, it was found that administering low dosages (less than 10,000 IU) of interferon beta-1a via oral or inhaled administration does not result in Th17 receptor desensitization and downregulation and instead effectively acts as a Th17 inhibitor. As a direct Th17 inhibitor, interferon beta-1a prevents and treats the damaging effects of COVID-19 inhibiting Th17 and its downstream cytokines. See the table in FIG. 2 reflecting these results.

To date, low dose oral or inhalation or nasal administration of interferon beta-1a has not been used specifically as a Th17 inhibitor. Further, it has not been used in low enough dosages when delivered orally or by inhalation or by nasal spray or drops so as not to downregulate the Th17 and IL-17 receptors in the intestinal and lung mucosa. Nor has it been understood in the art that dosages of interferons above 10,000 IU overwhelm the Th17 and IL-17 receptors in the intestinal and pulmonary mucosa because when systemically given, millions of IU are required for efficacy outcomes.

The present disclosure therefore utilizes interferon beta-1a for coronavirus illnesses including COVID-19, orally and by inhalation or by nasal spray or drops as a direct Th17 inhibitor delivering dosages orally or by inhalation or by nasal spray or drops directly to the Th17 receptor in the mucosal surfaces that are 1/10,000 to 1/100,000 (dosages ranging from 150 IU to 5,000 IU of interferon beta-1a are preferred) the dosages used for systemic intramuscular and subcutaneous injection.

Methods of dilution for interferon beta-1a include dilution in normal saline have been described using oral and inhaled interferons and include new formulations for oral delivery that optimize both the vehicle for oral delivery and method of delivery to reduce degradation of the active interferon beta-1a and include, but are not limited to a pill, capsule, tablet, chewable tablet, gum, chew, liquid, syrup, lozenge, dissolvable type formulations. Inhaled formulations are formulated to maintain efficacy and include use of nebulizer treatments and inhalation canisters (commonly referred to as inhalers).

In addition to administering a low dosage (orally or by inhalation or by nasal spray or drops) of interferon beta-1a as a Th17 inhibitor in the treatment coronavirus infections, including COVID-19, such methodology can be combined with the administration of antiviral pharmaceutical agents or drugs. For example, known antiviral drugs that can be combined with the low dosage interferon beta-1b include, but are not limited to, Ribavarin, Ritonavir, and Remdesivir.

Likewise, it has been shown that interferon beta-1a when administered at high dosages (for example, 1,000,000 IU to 6,000,000 IU or more), acts as a systemic anti-inflammatory agent. Thus, the disclosure also provides for a combination of administering dosages of less than 10,000 IU of interferon beta-1a orally and via inhalation or by nasal spray or drops as a direct Th17 inhibitor with a pharmaceutical agent or drug that has antiviral properties to modify the symptomatology of the disease state. See the Examples presented below for specific methodologies.

Thus the present disclosure describes the new finding that dosages higher than 10,000 IU of interferons (when administered orally or by inhalation or by nasal spray or drops) results in receptor desensitization and down regulation because interferons are being delivered directly to the Th17 and IL-17 receptors in the mucosal surface of the intestine. Further, the disclosure enables the use low dosage interferon beta-1a as a direct inhibitor of Th17, which is constitutively found in the intestinal mucosa and also found in the mucosa of the respiratory tract.

### EXAMPLES

This invention identifies methods of treating or preventing the effects of a coronaviruses infection including COVID-19. The methods include low doses of interferon beta-1a administered either orally or through inhalation or by nasal spray or drops. The methods also disclose using a Th17 inhibitor such as interferon beta-1a with or without an antiviral to treat or prevent the effects of a coronaviruses infection including COVID-19.

To that end, included herein are specific examples that describe how the inventions disclosed herein can be used in a clinical setting to treat or prevent the effects of a coronaviruses infection including COVID-19.

### Example 1

A patient who tests positive for COVID-19 with symptoms of cough and fever is given 5,000 IU of oral interferon beta-1a daily until he is both asymptomatic and he is no longer testing positive for COVID-19.

### Example 2

A patient who tests negative for COVID-19 who has a household member who is COVID-19 positive, begins 5,000 IU of oral interferon beta-1a in a chewable tablet form until his household member is asymptomatic and is no longer testing positive for COVID-19.

### Example 3

A patient who tests positive for COVID-19 with severe symptoms of cough and fever requiring hospitalization and has a pulse oximetric reading of 80% is given 5,000 IU by nebulizer daily until he is asymptomatic and no longer tests positive for COVID-19.

### Example 4

An 88-year old patient who has fever, malaise and body aches and is in a nursing home and has been potentially exposed to COVID-19, but the facility is unable to obtain testing to confirm that the patient is COVID-10 positive. His physician begins the patient on a liquid form oral interferon beta-1a in the equivalent strength of 5,000 IU daily until testing can be performed. If the testing is positive, the patient will remain oral interferon beta-1a until he tests negative for COVID-19.

### Example 5

A 45-year old woman is quarantined at home with a positive COVID-19 test. She has 5 others living in the household. Within 2 weeks, 2 members (under the age of 18) of the household (under the age of 18) develop symptoms cough and fever. The family physician encourages testing of the symptomatic family members, who do not drive. The household members have tried to distance themselves 6-feet from one another. The doctor begins the 2 symptomatic patients and all members of the household to begin 5,000 IU of interferon beta-1a and recommends calling an ambulance if any family experiences significant shortness of breath.

### Example 6

A 60 year old man presents with severe respiratory difficulties with a oxygen saturation of 80% and may need assisted ventilation who has tested positive for COVID-19, he is begun on 5,000 IU of oral interferon beta-1a and 6,000,000 IU intramuscularly of interferon beta 1a with both given daily until his oxygen saturation is above 90% at which time, his intramuscular injections of interferon beta-1a are reduced to weekly.

### Example 7

A 70 year old man presented with severe respiratory distress with an oxygen saturation of 70% and requires intubation. He tests positive for COVID-19. He is begun on the antiviral Ribavarin 600 mg orally twice daily, 5,000 IU oral interferon beta-1a, which is taken until he is extubated, asymptomatic with a negative COVID-19 test.

### Example 8

A 40 year old male presents with diffuse muscle aches, malaise and fever of 101 degrees Fahrenheit. He tests positive for COVID-10 and is begun on 5,000 IU of oral interferon beta-1a daily at bedtime and is also started on the antiviral Ritonavir in a dose escalation of 300 mg twice daily with an increase by 100 mg twice daily every to days and maintained at a dosage of 600 mg twice daily. He is maintained on both therapies until he is asymptomatic and has a negative COVID-19 test.

### Example 9

A 45 year old woman with recent history of surgery for breast cancer followed by chemotherapy, for which she has one more chemotherapy session in 2 weeks. Her cousin found out that she was COVID-19 positive today. The patient last saw her cousin one week ago and has no symptoms. Her doctor begins her on 500 IU of oral interferon alpha immediately and gives her a prescription and is told to call the scheduling phone number for a COVID-19 test

### Example 10

A 30 year old man is living at home with his wife who was just found COVID-19 positive and he is COVID-10 negative. His doctor prescribes both the patient and her husband 5,000 IU of oral interferon beta-1a

### Example 11

A 56 year old male with diabetes has sudden headache, fever and shortness of breath. He reports having played cards with friends 5 days ago, and 3 others have come down with the same symptoms. In his physician's office, he found to have an oxygen saturation of 86% with labored breathing. He is given a nebulizer treatment of 5,000 IU of interferon beta-1a in the office while awaiting transfer to the emergency room. In the ER, he receives daily inhaled interferon beta-1a in doses of 5,000 IU.

### Example 12

A 30 year very anxious mother of 4 children presents to her doctor having had a possible exposure to COVID-19 by one of neighbors who is now sick. She is extraordinarily concerned about getting any of the COVID-19 symptoms. Her doctor checks her for COVID-19 with results coming back in 5 days and begins her 5,000 IU of oral interferon beta-1 a.

### Example 13

A 30 year old man is living at home with his wife who was just found COVID-19 positive and he is COVID-10 negative. His doctor prescribes both the patient and her husband 5,000 IU of oral interferon beta-1a

I claim:

1. A method of treating or preventing the effects of a coronaviruses infection including COVID-19, the method comprising the step of:
administering a therapeutically effective amount orally or by inhalation or by nasal spray or drops a composition comprising a dosage of less than 10,000 IU of interferon beta-1 a.

2. The method of claim 1 wherein the dosage of interferon beta-1a in the composition is 5,000 IU or less.

3. A method of treating or preventing the effects of a coronaviruses infection including COVID-19, the method comprising the step of:
administering a therapeutically effective amount orally or by inhalation or by nasal spray or drops a composition comprising a Th17 inhibitor.

4. The method of claim 3 wherein the Th17 inhibitor is interferon beta-1a.

5. The method of claim 4 wherein the dosage of the Th17 inhibitor interferon beta-1a in the composition is less than 10,000 IU.

6. The method of claim 5 wherein the dosage of the Th17 inhibitor interferon beta-1a in the composition is 5,000 IU or less.

7. The method of claim 3, further comprising the step of:
administering a therapeutically effective amount of an antiviral.

8. The method of claim 7, wherein the antiviral is selected from the group consisting of Ribavarin, Ritonavir, and Remdesivir.

9. The method of claim 3, further comprising the step of:
administering a therapeutically effective amount of sarilumad.

10. The method of claim 3, further comprising the step of:
administering a therapeutically effective amount of Vitamin C.

11. A method of treating or preventing the effects of a coronaviruses infection including COVID-19, the method comprising the steps of:
administering a therapeutically effective amount orally or by inhalation or by nasal spray or drops a composition comprising a Th17 inhibitor; and
administering a therapeutically effective amount of an anti-inflammatory agent.

12. The method of claim 11, wherein the Th17 inhibitor is interferon beta-1a.

13. The method of claim 12, wherein the dosage of the Th17 inhibitor interferon beta-1a in the composition is less than 10,000 IU.

14. The method of claim 13, wherein the dosage of the Th17 inhibitor interferon beta-1a in the composition is 5,000 IU or less.

15. The method of claim 11, wherein the anti-inflammatory agent is administered systemically.

16. The method of claim 11 wherein the anti-inflammatory agent is interferon beta-1a.

17. The method of claim 12 wherein the dosage of the anti-inflammatory agent interferon beta-1a in the composition is at least 1,000,000 IU.

18. The method of claim 11, wherein the Th17 inhibitor is interferon beta-1a and the anti-inflammatory agent is interferon beta-1a.

19. The method of claim 18, wherein the dosage of the Th17 inhibitor interferon beta-1a in the composition is less than 10,000 IU, and wherein the dosage of anti-inflammatory agent interferon beta-1a is at least 1,000,000 IU.

20. The method of claim 19, wherein the dosage of the Th17 inhibitor interferon beta-1a in the composition is 5,000 IU or less, and wherein the dosage of anti-inflammatory agent interferon beta-1a is at least 1,000,000 IU.
